(19) United States
(12) Patent Application Publication
(10) Pub. No.: US 2025/0351842 A1
(43) Pub. Date: Nov. 20, 2025

(54) PREPARATION METHOD AND APPLICATION OF GINGER SEEDLING FERMENTED FEED FOR SOWS

(71) Applicants: SHANDONG AGRICULTURAL UNIVERSITY, Tai'an (CN); SHANDONG ZHONGCHENG FEED TECHNOLOGY CO., LTD, Tai'an (CN)

(72) Inventors: Shuzhen JIANG, Tai'an (CN); Mengjun WANG, Tai'an (CN); Lijie YANG, Tai'an (CN); Weiren YANG, Tai'an (CN)

(21) Appl. No.: 19/015,984

(22) Filed: Jan. 10, 2025

(30) Foreign Application Priority Data
May 17, 2024 (CN) 202410615299.5

(51) Int. Cl. A23K 10/12; A23K 10/30; A23K 20/142; A23K 20/174; A23K 20/20; A23K 20/22; A23K 20/26; A23K 50/30; A61K 36/9068

(52) U.S. Cl. CPC A23K 10/12; A23K 10/30; A23K 20/142; A23K 20/174; A23K 20/22; A23K 20/26; A23K 20/30; A23K 50/30; A61K 36/9068; A23V 2400/41; A61K 2236/15; A61K 2236/19

(57) ABSTRACT

A preparation method and application of ginger seedling fermented feed for sows is provided. The preparation method includes steps of: crushing and pressurizing ginger seedlings to obtain crushed ginger seedling material and ginger seedling juice; adding bran into the crushed ginger seedling material to adjust the moisture content, adding compound bacteria and compound enzyme, and performing anaerobic fermentation to obtain ginger seedling biological feed; adding molasses and compound bacteria into the ginger seedling juice, and performing anaerobic fermentation to obtain fermented ginger seedling juice; using the fermented ginger seedling juice to adjust a moisture content of a basic diet, and performing anaerobic fermentation to obtain fermented ginger seedling juice concentrated feed; and uniformly mixing the ginger seedling biological feed, the fermented ginger seedling juice concentrated feed and premix to obtain the ginger seedling fermented feed for sows.

### CROSS-REFERENCE TO RELATED APPLICATIONS

This application claims priority of Chinese Patent Application No. 202410615299.5, filed on May 17, 2024, the entire contents of which are incorporated herein by reference.

### TECHNICAL FIELD

The present disclosure belongs to the technical field of feed preparation, and in particular to a preparation method and application of ginger seedling fermented feed for sows.

### BACKGROUND

Recently, fermented feed has attracted more and more attention because of its palatability, digestibility, feed source and feed cost. Feed fermentation can not only convert anti-nutritional molecules into non-toxic components, but also convert macromolecular substances in feed into small molecular substances which are beneficial to intestinal absorption of animals, and can increase the concentration of beneficial bacteria, enzymes and metabolites in feed.

In animal husbandry, the management of sow breeding is very important to improve the breeding efficiency. With the continuous development of aquaculture technology, fermented feed has been widely used in animal husbandry because of its unique advantages and improving the nutritional value of feed. However, although the common fermented feed on the market at present improves the nutritional value of feed to a certain extent, it often leads to a decrease in feed intake rate because of its poor palatability.

Therefore, how to improve the palatability of feed, improve the feed intake rate, and then promote the health and reproductive performance of sows is an urgent problem for technicians in this field.

### SUMMARY

The objective of the present disclosure is to provide a preparation method and application of ginger seedling fermented feed for sows, so as to solve the problems existing in the prior art.

In order to achieve the above objective, the present disclosure provides the following scheme.

One of the technical schemes of the disclosure is to provide a preparation method of ginger seedling fermented feed for sows, which includes following steps:

S1, crushing and pressurizing ginger seedlings to obtain crushed ginger seedling material with moisture content of 70-75% and ginger seedling juice;

S2, adding bran into the crushed ginger seedling material to adjust the moisture content to 60-65%, adding compound bacteria and compound enzyme, and performing anaerobic fermentation for 7-14 days to obtain ginger seedling biological feed;

S3, adding molasses and compound bacteria into the ginger seedling juice, and performing anaerobic fermentation for 7 days to obtain fermented ginger seedling juice;

S4, using the fermented ginger seedling juice to adjust the moisture content of a basic diet to 40-45%, and performing anaerobic fermentation for 4-5 days to obtain fermented ginger seedling juice concentrated feed; and

S5, uniformly mixing the ginger seedling biological feed, the fermented ginger seedling juice concentrated feed and premix to obtain the ginger seedling fermented feed for sows. Optionally, a moisture content of the ginger seedlings is 85-90%.

Optionally, a particle size of the crushed ginger seedling material is 1-10 mm.

Optionally, in the S2, a mass ratio of the crushed ginger seedling material, the compound enzyme and the compound bacteria is 100:0.1-1:0.1-1.

Optionally, in the S3, the mass ratio of ginger seedling juice, molasses and compound bacteria is 100:2-5:0.1-1.

Optionally, the basic diet includes, by mass, 70 parts of corn and 25 parts of soybean meal.

Optionally, each 50 g of the premix includes: calcium hydrogen phosphate 0.8 g, salt 0.17 g, amino acid 1 g, VA 7700 IU, VD₃ 3400 IU, VE 30 IU, VB₆ 4.0 mg, VB₁₂ 0.05 mg, nicotinic acid 36 mg, pantothenic acid 28 mg, biotin 0.32 mg, folic acid 5 mg, iron 120 mg, manganese 100 mg, copper 26 mg, zinc 95 mg, iodine 0.2 mg and selenium 0.4 mg.

Optionally, in the S5, in percentage of 100% of the ginger seedling fermented feed for sows by mass, a mass percentage of the ginger seedling biological feed is 30-50%, a mass percentage of the fermented ginger seedling juice concentrated feed is 50-65%, and a mass percentage of the premix is 5%.

Optionally, the compound bacteria is composed of Pediococcus lactis, Saccharomyces cerevisiae and Bacillus subtilis in equal mass ratio.

Among them, the bacterial content of Pediococcus lactis, Saccharomyces cerevisiae or Bacillus subtilis is not less than 5×10⁸ cfu/g.

Optionally, the compound enzyme includes pectinase, cellulase and hemicellulase, and their mass ratios are equal.

Among them, the enzyme activity of pectinase, cellulase or hemicellulase is not less than 100 u/g.

Optionally, the temperature of the anaerobic fermentation is not higher than 50° C.

Another technical scheme of the disclosure is to provide a ginger seedling fermented feed for sows prepared by above preparation method.

Another technical scheme of the disclosure is to provide an application of the ginger seedling fermented feed for sows in improving the feed intake of sows, relieving the constipation of sows, improving the resistance of sows and accelerating the postpartum recovery of sows.

The disclosure discloses the following technical effects.

Ginger seedlings are added into the raw materials of ginger seedling fermented feed for sows, crushed ginger seedling material and ginger seedling juice are obtained through crushing and pressurizing, and the crushed ginger seedling material and ginger seedling juice are fermented respectively to obtain ginger seedling biological feed and fermented ginger seedling juice concentrated feed, and the ginger seedling juice is further used for fermenting corn and soybean meal in the basic diet, so that the utilization rate of ginger seedlings is improved, and the nutritional value of the basic diet is improved through fermentation, which makes it easier to digest.

According to the disclosure, through enzymolysis and anaerobic fermentation, the nutrient components in the ginger seedlings are better released and utilized, where the temperature of enzymolysis and fermentation are controlled within 50° C., so the activity of gingerol in the ginger seedlings is guaranteed, and the nutritional value of fermented feed is improved.

The ginger seedling fermented feed for sows prepared by the disclosure inherits gingerol, namely volatile oil and other substances contained in ginger seedlings, and has the effects of promoting digestive juice secretion, improving appetite, promoting body metabolism and the like, thereby improving the feed intake of sows, relieving constipation of sows and improving the resistance of sows; good nutritional support has an important influence on postpartum recovery and lactation health of sows, and rich plant fiber can ensure smooth defecation without constipation after eating.

### DETAILED DESCRIPTION OF THE EMBODIMENTS

A number of exemplary embodiments of the present disclosure will now be described in detail, and this detailed description should not be considered as a limitation of the present disclosure, but should be understood as a more detailed description of certain aspects, characteristics and embodiments of the present disclosure.

It should be understood that the terminology described in the present disclosure is only for describing specific embodiments and is not used to limit the present disclosure. In addition, for the numerical range in the present disclosure, it should be understood that each intermediate value between the upper limit and the lower limit of the range is also specifically disclosed. Intermediate values within any stated value or stated range, as well as each smaller range between any other stated value or intermediate values within the stated range are also included in the present disclosure. The upper and lower limits of these smaller ranges can be independently included or excluded from the range.

Unless otherwise specified, all technical and scientific terminology used herein have the same meaning as commonly understood by one of ordinary skill in the art to which this disclosure relates. Although the present disclosure only describes the preferred methods and materials, any methods and materials similar or equivalent to those described herein may also be used in the practice or testing of the present disclosure. All documents mentioned in this specification are incorporated by reference to disclose and describe methods and/or materials related to the documents. In case of conflict with any incorporated document, the contents of this specification shall prevail.

It is obvious to those skilled in the art that many improvements and changes may be made to the specific embodiments of the present disclosure without departing from the scope or spirit of the present disclosure. Other embodiments will be apparent to the skilled person from the description of the disclosure. The description and example of that present disclosure are exemplary only.

The terms “consisting”, “including”, “having” and “containing” used in this article are all open terms, which means including but not limited to.

The raw materials used in the disclosure are all commercially available products, and the purchase channel does not affect the realization of technical effects.

Unless otherwise specified, in the specific embodiment of the present disclosure, the term room temperature or normal temperature refers to 20° C.-30° C.

In the specific embodiment of the disclosure, the particle size of the crushed ginger seedling material is between 1 and 10 mm, including endpoint values.

In the specific embodiment of the disclosure, the compound enzyme includes pectinase, cellulase and hemicellulase, and their mass ratios are equal, and the enzyme activity is not less than 100 u/g; the compound bacteria is composed of Pediococcus lactis, Saccharomyces cerevisiaeand Bacillus subtilis in equal mass ratio, where the content of Pediococcus lactis is 1.5×10⁹ cfu/g, the content of Saccharomyces cerevisiae is 3×10⁹ cfu/g, and the content of Bacillus subtilis is 5×10⁸ cfu/g. The above-mentioned compound enzyme and compound bacteria are commercially available products.

In the specific embodiment of the disclosure, the basic diet includes, by mass, 70 parts of corn and 25 parts of soybean meal; each 50 g of the premix includes: calcium hydrogen phosphate 0.8 g, salt 0.17 g, amino acid 1 g, VA 7700 IU, VD₃ 3400 IU, VE 30 IU, VB₆ 4.0 mg, VB₁₂ 0.05 mg, nicotinic acid 36 mg, pantothenic acid 28 mg, biotin 0.32 mg, folic acid 5 mg, iron 120 mg, manganese 100 mg, copper 26 mg, zinc 95 mg, iodine 0.2 mg and selenium 0.4 mg.

### Embodiment 1

The preparation of ginger seedling fermented feed for sows includes following steps:

S1, crushing and pressurizing ginger seedlings (moisture content of 90%) to obtain crushed ginger seedling material with moisture content of 75% and ginger seedling juice;

S2, adding bran into the crushed ginger seedling material to adjust the moisture content to 60%, adding compound enzyme and compound bacteria, and performing anaerobic fermentation for 14 days (the temperature is not higher than 50° C.) to obtain ginger seedling biological feed, where the mass ratio of the crushed ginger seedling material, compound enzyme and compound bacteria is 100:1:1;

S3, adding molasses and compound bacteria into the ginger seedling juice, and performing anaerobic fermentation for 7 days (the temperature is not higher than 50° C.) to obtain fermented ginger seedling juice, where the mass ratio of the ginger seedling juice, molasses and compound bacteria is 100:3:0.5;

S4, using the fermented ginger seedling juice to adjust the moisture content of corn and soybean meal of the basic diet to 45%, and performing anaerobic fermentation for 5 days (the temperature is not higher than 50° C.) to obtain fermented ginger seedling juice concentrated feed; and

S5, uniformly mixing the ginger seedling biological feed, the fermented ginger seedling juice concentrated feed and premix according to mass ratio of 30:65:5 to obtain the ginger seedling fermented feed for sows.

### Embodiment 2

The preparation of ginger seedling fermented feed for sows includes following steps:

S1, crushing and pressurizing ginger seedlings (moisture content of 90%) to obtain crushed ginger seedling material with moisture content of 70% and ginger seedling juice;

S2, adding bran into the crushed ginger seedling material to adjust the moisture content to 60%, adding compound enzyme and compound bacteria, and performing anaerobic fermentation for 7 days (the temperature is not higher than 50° C.) to obtain ginger seedling biological feed, where the mass ratio of the crushed ginger seedling material, compound enzyme and compound bacteria is 100:1:1;

S3, adding molasses and compound bacteria into the ginger seedling juice, and performing anaerobic fermentation for 7 days (the temperature is not higher than 50° C.) to obtain fermented ginger seedling juice, where the mass ratio of the ginger seedling juice, molasses and compound bacteria is 100:2:1;

S4, using the fermented ginger seedling juice to adjust the moisture content of corn and soybean meal of the basic diet to 40%, and performing anaerobic fermentation for 4 days (the temperature is not higher than 50° C.) to obtain fermented ginger seedling juice concentrated feed; and

S5, uniformly mixing the ginger seedling biological feed, the fermented ginger seedling juice concentrated feed and premix according to mass ratio of 30:65:5 to obtain the ginger seedling fermented feed for sows.

### Embodiment 3

The preparation of ginger seedling fermented feed for sows includes following steps:

S1, crushing and pressurizing ginger seedlings (moisture content of 90%) to obtain crushed ginger seedling material with moisture content of 70% and ginger seedling juice;

S2, adding bran into the crushed ginger seedling material to adjust the moisture content to 65%, adding compound enzyme and compound bacteria, and performing anaerobic fermentation for 10 days (the temperature is not higher than 50° C.) to obtain ginger seedling biological feed, where the mass ratio of the crushed ginger seedling material, compound enzyme and compound bacteria is 100:0.5:0.5;

S3, adding molasses and compound bacteria into the ginger seedling juice, and performing anaerobic fermentation for 7 days (the temperature is not higher than 50° C.) to obtain fermented ginger seedling juice, where the mass ratio of the ginger seedling juice, molasses and compound bacteria is 100:5:0.1;

S4, using the fermented ginger seedling juice to adjust the moisture content of the basic diet to 45%, and performing anaerobic fermentation for 5 days (the temperature is not higher than 50°° C.) to obtain fermented ginger seedling juice concentrated feed; and

S5, uniformly mixing the ginger seedling biological feed, the fermented ginger seedling juice concentrated feed and premix according to mass ratio of 30:65:5 to obtain the ginger seedling fermented feed for sows.

### Comparative Example 1

The preparation steps of fermented feed are as follows:
crushing ginger seedlings (moisture content is 90%), then adding basic diet to adjust the moisture content to 45%, then adding compound enzyme and compound bacteria, and performing anaerobic fermentation for 14 days (the temperature is not higher than 50° C.), followed by mixing with premix to obtain fermented feed, where the mass ratio of ginger seedling, compound enzyme and compound bacteria is 100:1:1.

### Comparative Example 2

The preparation steps of fermented feed are as follows:
adjusting the moisture content of corn and soybean meal in the basic diet to 45% with water, adding compound bacteria, performing anaerobic fermentation for 5 days (the temperature is not higher than 50°° C.), and then mixing with premix evenly to obtain fermented feed, where the mass ratio of basic diet, premix and compound bacteria is 94:5:1.

### Comparative Example 3

The preparation of ginger seedling fermented feed for sows includes following steps:

S1, crushing and pressurizing ginger seedlings (moisture content of 90%) to obtain crushed ginger seedling material with moisture content of 75% and ginger seedling juice;

S2, adding bran into the crushed ginger seedling material to adjust the moisture content to 60%, adding compound enzyme and compound bacteria, and performing anaerobic fermentation for 14 days (the temperature is not higher than 50° C.) to obtain ginger seedling biological feed, where the mass ratio of the crushed ginger seedling material, compound enzyme and compound bacteria is 100:1:1;

S3, adding molasses and compound bacteria into the ginger seedling juice, and performing anaerobic fermentation for 7 days (the temperature is not higher than 50°° C.) to obtain fermented ginger seedling juice, where the mass ratio of the ginger seedling juice, molasses and compound bacteria is 100:3:0.5;

S4, using the fermented ginger seedling juice to adjust the moisture content of the corn and soybean meal of the basic diet to 45% to obtain fermented ginger seedling juice mixture; and

S5, uniformly mixing the ginger seedling biological feed, the fermented ginger seedling juice mixture and premix according to mass ratio of 30:65:5 to obtain the ginger seedling fermented feed for sows.

### Experiment Example

Forty-two pregnant sows with similar parity, weight and expected date of delivery are selected and divided into seven groups with six sows in each group. The cows in the control group are fed with mixture of basic diet and water, where the ratio of water to feed is 1.6:1. The cows in the treatment group are subjected to gruel feeding, namely fed with mixture of feed in Embodiments 1-3 or Comparative Examples 1-3 and water, where the ratio of water to feed is 1.6:1. The experiment is carried out from 100 days of pregnancy to 30 days after delivery, and the sows are free to eat and drink water. The weight of sows at the beginning of the experiment and at the end of the experiment, as well as the average daily feed intake, are recorded. The results are shown in Table 1.

TABLE 1

| | Average daily | | Weight (kg) | | |
|---|---|---|---|---|---|
| | feed intake | | 100 days | On the | 30 days |
| Experiment | Before | After | of | day of | after |
| groups | delivery | delivery | pregnancy | delivery | delivery |
| Embodiment 1 | 2.7 | 4.0 | 251 | 215 | 233 |
| Embodiment 2 | 2.6 | 3.9 | 250 | 214 | 230 |
| Embodiment 3 | 2.8 | 4.2 | 251 | 218 | 232 |
| Comparative | 2.3 | 3.2 | 249 | 208 | 221 |
| Example1 | | | | | |
| Comparative | 2.0 | 3.1 | 251 | 212 | 220 |
| Example2 | | | | | |
| Comparative | 2.5 | 3.8 | 250 | 209 | 227 |
| Example3 | | | | | |
| Control group | 2.5 | 3.6 | 249 | 210 | 225 |

As can be seen from the data in Table 1, in Comparative Example 1, the ginger seedlings are crushed and mixed with the basic diet as a whole for anaerobic fermentation. Because the basic diet undergoes fermentation for too long time, the taste is affected, resulting in a decrease in the feed intake. However, in Embodiments 1 to 3 of the present disclosure, ginger seedlings are added, and the ginger seedling crushed material and ginger seedling juice are subjected to anaerobic fermentation respectively. In this way, both the taste of the feed and the daily feed intake are improved compared with the control group, thus increasing the total daily nutrition intake of sows; meanwhile, the nutritional supply in the third trimester and postpartum are also ensured, making weight recovery better after delivery. Through the weight change, it can be further found that the postpartum weight recovery of cows in the groups fed with the feed of Embodiments 1 to 3 of the present disclosure is better than that of cows in the Comparative Examples 1 to 3 and the control group, indicating that the feed provided by the present disclosure not only provides sufficient nutrition, but also promotes the recovery of sows after farrowing. In Comparative Example 2, although the digestibility of the basic diet could be improved, food intake is low because only basic diet is fermented and it does not contain gingerol components in ginger seedlings and effective components to promote sow's intake. In Comparative Example 3, the basic diet is not fermented, and its palatability is poor, and the digestibility of the unfermented diet is worse than that of the groups in Embodiments 1 to 3 of the present disclosure.

The above description of the disclosed embodiments enables those skilled in the art to implement or use the disclosure. Many modifications to these embodiments will be obvious to those skilled in the art, and the general principles defined herein can be implemented in other embodiments without departing from the spirit or scope of the disclosure. Therefore, the present disclosure is not to be limited to the embodiments shown herein, but is to be accorded the widest scope consistent with the principles and novel features disclosed herein.

1. A preparation method of ginger seedling fermented feed for sows, comprising following steps:
S1, crushing and pressurizing ginger seedlings to obtain crushed ginger seedling material with a moisture content of 70-75% and ginger seedling juice;
wherein a moisture content of the ginger seedlings is 85-90%, and a particle size of the crushed ginger seedling material is 1-10 mm;
S2, adding bran into the crushed ginger seedling material to adjust the moisture content to 60-65%, adding an inoculum composed of Pediococcus lactis, Saccharomyces cerevisiae and Bacillus subtilis in equal mass ratio and compound enzyme comprising pectinase, cellulase and hemicellulase in equal mass ratio, and performing anaerobic fermentation for 7-14 days at a temperature not higher than 50°° C. to obtain ginger seedling biological feed;
wherein the mass ratio of the crushed ginger seedling material, the compound enzyme and the inoculum is 100:0.1-1:0.1-1;
S3, adding molasses and inoculum into the ginger seedling juice, and performing anaerobic fermentation for 7 days to obtain fermented ginger seedling juice;
wherein the mass ratio of the ginger seedling juice, the molasses and the inoculum is 100:2-5:0.1-1;
S4, using the fermented ginger seedling juice to adjust the moisture content of a basic diet to 40-45%, and performing anaerobic fermentation for 4-5 days to obtain fermented ginger seedling juice concentrated feed; and
S5, uniformly mixing the ginger seedling biological feed, the fermented ginger seedling juice concentrated feed and premix to obtain the ginger seedling fermented feed for sows;
wherein in percentage of 100% of the ginger seedling fermented feed for sows by mass, the mass percentage of the ginger seedling biological feed is 30-50%, the mass percentage of the fermented ginger seedling juice concentrated feed is 50-65%, and the mass percentage of the premix is 5%.

2. The preparation method according to claim 1, wherein the basic diet comprises, by mass, 70 parts of corn and 25 parts of soybean meal.

3. The preparation method according to claim 1, wherein each 50 g of the premix comprises: calcium hydrogen phosphate 0.8 g, salt 0.17 g, amino acid 1 g, VA 7700 IU, VD₃ 3400 IU, VE 30 IU, VB₆ 4.0 mg, VB₁₂ 0.05 mg, nicotinic acid 36 mg, pantothenic acid 28 mg, biotin 0.32 mg, folic acid 5 mg, iron 120 mg, manganese 100 mg, copper 26 mg, zinc 95 mg, iodine 0.2 mg and selenium 0.4 mg.

4. (canceled)

5. A ginger seedling fermented feed for sows prepared by the preparation method according to claim 1.
